(19) United States
(12) Patent Application Publication
(10) Pub. No.: US 2011/0015499 A1
(43) Pub. Date: Jan. 20, 2011

(54) PATIENT BED WITH MONITORING AND THERAPY DEVICE

(76) Inventors: Clemens Gutknecht, Konstanz (DE); Josef Ludwig Schoenberger, Amriswil (CH)

Correspondence Address: PAI PATENT & TRADEMARK LAW FIRM, 1001 FOURTH AVENUE, SUITE 3200, SEATTLE, WA, 98154, US

(21) Appl. No.: 12/811,698

(22) PCT Filed: Jan. 5, 2009
(86) PCT No.: PCT/EP09/50030

§ 371 Date: Sep. 13, 2010

Related U.S. Application Data
(60) Provisional application No. 61/026,140, filed on Feb. 5, 2008.

(30) Foreign Application Priority Data
Jan. 3, 2008 (CH) 00006/08

(51) Int. Cl. A61G 7/015; A61G 7/002; A61G 7/005; A61B 5/02; A61B 5/01; A61G 15/00

(52) U.S. Cl. 600/301; 5/600; 5/610; 5/613; 128/845

(57) ABSTRACT

The sick bed (1) can be rotated about an axis (9) in a direction indicated by an arrow (P), and consequently propped up. A patient strapped to the bed (4) will likewise be propped up. The verticalisation of the patient has effect on the physiological parameters of the same. According to the invention, the angle of inclination of the bed (4) relative to the horizontal is set according to the measured physiological parameters of the patient fixed on the bed (4).

### FIELD OF THE INVENTION

The present invention relates to a patient bed, comprising a frame, a bed connected to the frame and having a support surface for a patient, with at least one subsection of the bed arranged such that it can be moved relative to the frame, and at least one drive for setting the relative orientation of the at least one subsection of the bed relative to the frame. The invention includes a monitoring and therapy device, comprising: at least one patient bed, comprising a frame, a bed connected to the frame and having a support surface for a patient, with at least one subsection of the bed arranged such that it can be moved relative to the frame, and at least one drive for setting the relative orientation of the at least one subsection of the bed relative to the frame.

### PRIOR ART

In the acute phase of a number of clinical pictures, substantial dysregulation of, e.g. heart rate and blood pressure (“high” and/or “low”) occurs within a few days that can be due on one hand to the primary disease process, and on the other to confinement to bed. From the acute medical point of view, one of the key therapy principles consists in attaining lasting normalisation of these physiological parameters early in the acute phase of the illness. For example, in an acute stroke, standard parameters or values to be prescribed by the attending physician for blood pressure, heart rate, blood sugar, body temperature, intracranial pressure and other physiological measured values should be attained as early as possible, as this is of major importance for successful treatment.

These targets are reached in conventional treatment primarily by the administration of drugs, but also by the application of physical measures (e.g. cold/heat applications, compression therapy of the legs, mobilisation physiotherapy, etc.). One problem, however, is the issue of the intensity or dosage of the application. Another is that the individual measures have various side effects.

Besides these measures, it is possible to verticalise the patient, that is, prop him or her up. This principle is employed in the care and therapy of bedridden patients, e.g. with the help of “standing frames”, “standing beds” etc., with the latter, in principle, being semi-automated, motorised standing frames, the advantage of which is that the patient's position does not have to be changed.

Semi-automated mobilisation of bedridden patients is possible in a variety of ways, too, e.g. by means of motorised leg or arm braces. Motorised, semi-automated or robot-assisted therapy and training devices are also already used in medicine, e.g. in the form of treadmills.

However, usually the impact of all of the above measures cannot be captured in real time.

### OBJECT OF THE INVENTION

Proceeding therefrom, the object of the present invention is to provide a patient bed or a combination of it and a monitoring and therapy device which allows treatment for achieving targeted normalisation and control of a patient's physiological parameters by physical active variables.

### TECHNICAL SOLUTION

This object is achieved by a patient bed in accordance with claim 1 and by a monitoring and therapy device in accordance with claim 10.

The inventive patient bed comprises a frame, a bed connected to the frame and having a support surface for a patient, with at least one subsection of the bed arranged such that it can be moved relative to the frame, and at least one drive for setting the relative orientation of the at least one subsection of the bed relative to the frame. The patient bed has at least one controller and/or regulator for activating the at least one drive as a function of at least one measured physiological parameter of the patient.

In the context of the invention, patient bed is understood to mean every type of sick bed, such as intensive-care care bed, nursing bed, acute care bed, and so on. The bed basically has a conventional construction comprising a support surface (e.g., slats or similar) upon which a mattress is arranged. The bed frame supports the support surface and can be provided with a restraint that at least partially surrounds the support surface to prevent the patient from falling out of the bed. In this way, an apparatus is provided that also permits long-term confinement of the patient. The use of the apparatus is thus not limited by time to a specific application, as may happen in the case of pure therapy devices. The inventive patient bed thus facilitates the realisation of a combined monitoring and therapy concept which permits almost real-time control over physiological parameters, such as blood pressure, heart rate, intracranial pressure, etc., in bedridden patients in the acute and/or rehabilitation phase. The concept is suitable for patients requiring monitoring in intensive care, in rehabilitation facilities, for patients suffering from stroke, heart attack, traumatic brain injury, paraplegia, intracranial pressure, or recovering from major surgery. The invention makes use of the concept of a patient bed or intensive-care bed which is amenable to regulation and control by physiological parameters. The physical/physiological input parameters and/or the parameters to be regulated can include measurement or regulation of intracranial pressure, blood pressure, heart rate, body temperature, blood sugar, cutaneous circulation, sweat regulation, basal metabolic rate, etc. The assistance of the patient bed facilitates personalised, doctor-directed, automated and continuous control/regulation of physiological patient parameters.

The control/regulation makes for reliable treatment success (in the sense of maintaining physiological parameters within a medically-defined range), which especially can also be monitored. Corresponding software and hardware can be provided for the regulation.

In the case of the inventive patient bed, the passive support surface or bed itself of known concepts boasts electromechanical equipment which transforms it into an active, medical, nursing and therapeutic “instrument” in the sense of a “micro-environment” (a system that regulates a patient's homeostasis). As a result, comprehensive monitoring is possible. The feedback controller facilitates continuous regulation and control of important physiological measured values and loads in the prophylaxis and treatment of bedridden patients.

It is preferred that the at least one drive is configured for setting the inclination of the support surface of the bed or a subsection of the support surface of the bed relative to the frame.

The at least one drive can be configured to change in particular the orientation of a part of the support surface of the bed for the purpose of mobilising a body part of the patient lying on the support surface.

Setting of the orientation of the bed, in particular its inclination relative to the frame, or the movement of individual areas of the support surface for the purpose of mobilising certain parts of the body of the patient proceeds with the help of the drive. This includes one or more motors, especially one or more electric motors, which are actuated by the controller/regulator and can set and change the position of the bed. The positioning unit, which permits positioning and movement of individual limbs (legs, arms, head/upper body) or rotation of the whole body, also serves the purpose of regular repositioning of bedridden patients to prevent pressure sores. The mobilising unit is configured for mobilising individual limbs, including independently of each other.

With the assistance of the change in the inclination of the support surface relative to the frame, a patient can be verticalised. In particular, provision is made in the context of the invention for dynamic (feed-back controlled) verticalisation. The angle of inclination in this connection can be approximately 90° relative to the horizontal or relative to the carrier, generally between 0° and 90° (with “negative” verticalisation, i.e. head down or upper-body down, being expressly possible, too). The angle is dynamically controlled as a function of the input variables. As a rule, the patient is securely strapped to the support surface, possibly supported by a foot section. Regardless of the extent of verticalisation of the support surface, the bed as a whole can be adjusted in its height, as a result of which (nursing, medical, therapeutic) measures can be performed on the patient at any time. The verticalisation unit thus permits on-demand verticalisation to almost 90 degrees, or even negative verticalisation (head down or upper-body down positions). Modified height adjustment of the overall bed facilitates work on the patient.

Verticalisation and passive mobilisation by means of motorised changes in the orientation of the patient relative to the horizontal and/or mobilisation of certain parts of the patient's body are a helpful way of positively influencing the recovery process. Heart rate and blood pressure and other physiological parameters can be regulated and controlled in almost real time (in the range of seconds or minutes) as a function of verticalisation angle and leg movement frequency and other physical parameters. With the help of the patient bed, it is possible to confine a non-mobile patient during a prolonged illness or recovery phase while simultaneously mobilising and monitoring the patient in a controlled manner for part or all of the period in the bed. For monitoring purposes, an acoustic and/or optical monitoring device can be provided that indicates the condition of the patient and the mobilisation measures carried out.

The patient bed preferably comprises a measuring device for continuous measurement of at least one physiological parameter of the patient, especially circulation parameters, such as heart rate, blood pressure, pulse, as well as body temperature, intracranial pressure, etc. Where possible, the patient parameters are monitored continuously, otherwise they are monitored at discrete, albeit relatively short time intervals. The measured values are fed into the controller/regulator and evaluated there. The patient parameters can also be measured continuously. The measuring device performs a monitoring function in addition to the measuring function. The measurement and monitoring unit continuously measures and documents heart rate, blood pressure (pulse wave transit time) and, after a corresponding probe has been surgically applied, the intracranial pressure as well. It can furthermore also comprise an alarm function for drawing attention to predetermined parameters which have been under-/over-exceeded. The measurement and control principle can be extended to all conceivable physiological parameters.

Especially, the controller/regulator has a control unit, a simple regulator, and/or an automatic regulator. The control and feedback unit allows individual physiological parameters of the patient (e.g. blood pressure, pulse, intracranial pressure, cerebral blood flow, cutaneous circulation, body temperature, body weight, sweat and excretion regulation, etc.) to be controlled by physical influences.

Preferably, the controller/regulator is arranged at the patient bed. The controller/regulator is therefore integrated into the bed. However, it can in principle also be provided separately from the patient bed, with the drives being actuated via a cable or wirelessly. The same applies to the measuring device that measures the patient's physiological parameters. The measured values can be transmitted either direct to the controller/regulator via a cable or wirelessly, both direct and via an intermediate processing unit.

The patient bed can be equipped such that the support surface can be cooled, warmed, put into a harder or softer setting, and/or rendered capable of conveying sensory stimuli. To this end, a heating device, a cooling device, a device for generating vibrations as a sensory stimulus, etc., can be provided in accordance with requirements. Alone or in combination with the verticalisation and/or mobilisation device, the positive effects of verticalisation or mobilisation can be supported or optimised by the aforementioned measures.

In addition, a stimulation unit can be provided that allows a patient to be exposed to specific stimuli (auditory, visual, olfactory, tactile, etc.), particularly to support or optimise the effects of verticalisation and/or mobilisation.

The object is also achieved by provision of a monitoring and therapy device, comprising: at least a patient bed, comprising a frame, a bed connected to the frame and having a support surface for a patient, with at least one subsection of the bed arranged such that it can be moved relative to the frame, and at least one drive for setting the relative orientation of the at least one subsection of the bed relative to the frame; and a controller/regulator for actuating the at least one drive as a function of at least one measured physiological parameter of the patient.

The bed is preferably connected to the frame such that the support surface can be inclined relative to the frame up to a predetermined angle. The angle is generally between 0° and 90°, but can also have negative values (usually 0° to −30°).

The at least one drive is configured in particular to change the orientation of a part of the support surface of the bed for the purpose of mobilising a body part of the patient lying on the support surface.

The inclination can be adjusted to an angle between −90° and +90°, especially an angle between about −30° and roughly +90° relative to the horizontal position of the support surface.

The inclination of the support surface can thus be adjustable such that the head or upper-body area of the bed is inclined or facing downwards. This feature is especially expedient for using the patient bed in a combined monitoring and therapy concept that permits almost real-time control over physiological parameters, such as blood pressure, heart rate, intracranial pressure, etc., in bedridden patients in the acute and/or rehabilitation phase, as in this way a number of physiological parameters can be influenced “in both directions.”

For example, the drive can comprise one or more hydraulic cylinders that can change the inclination of the bed or change the shape of the support surface.

The monitoring and therapy device preferably includes a measuring device for measuring the at least one physiological parameter of the patient, particularly a circulation parameter, such as heart rate, blood pressure, pulse, as well as body temperature, intracranial pressure, etc. The goal of the inventive device is maintenance of the homeostasis of bedridden patients in acute medical care, especially those in intensive care, in the sense of providing physical/physiological feedback.

Suitable measuring or sensor devices are known. Particularly interesting in respect of achieving the goal of continuous measurements are rapid measuring methods, which continuously determine the required measured values more or less instantaneously at short time intervals. Thus, non-invasive measurement of blood pressure based on the pulse wave transit time can be deployed. In this connection, the pressure fluctuations caused by pulse waves are measured continuously by means of a pressure transducer. From these, the systolic and diastolic blood pressure can be calculated using a mathematical algorithm.

Moreover, this application also claims a therapy concept for the human body, comprising the following steps: a) measurement of at least one physiological parameter of a patient, in particular by a measuring device, and b) mobilisation of a body part of a patient and/or change in the orientation of the patient's body as a function of the measured physiological parameter(s).

The type and intensity of mobilisation (e.g. leg or foot mobilisation) and/or the angle of inclination of the patient's body relative to the horizontal lying position proceeds in the context of regulation based on the measured physiological parameter(s).

As a rule, to this end, a drive for setting the inclination of the support surface of the bed or a subsection of the support surface of the bed is activated relative to the frame. The at least one drive can be configured in particular for changing the orientation of a part of the support surface of the bed for the purpose of mobilising an area of the patient's body lying on the support surface. The verticalisation can be in the range −90° and +90°, preferably between −30° and 90°. Negative inclinations correspond to a lowering of the head area relative to a foot area.

The physiological parameters (as actual values) are measured continuously or at discrete time intervals and compared with predetermined setpoint values. The deviation between the actual and the setpoint values determines the extent to which the type and/or the intensity of the mobilisation or the angle of inclination of the body is changed relative to the horizontal, i.e. increased or decreased. In this regard, the drives are controlled such that the deviation, as is usual in a regulator, is counteracted.

The therapy concept thus comprises preferably the step of an actual/setpoint comparison of the measured physiological parameter with a predetermined value.

Protection is sought for the described characteristics both individually and in any combination.

### BRIEF DESCRIPTION OF THE FIGURES

Other features and advantages of the invention are apparent from the description of preferred embodiments according to the attached Figures. They show in

FIG. 1 a first embodiment of an inventive sick bed;

FIG. 2 a second embodiment of an inventive sick bed; and

FIG. 3 a schematic illustration of the control circuit used in the invention.

### DESCRIPTION OF PREFERRED EMBODIMENTS

FIG. 1 shows an inventive sick bed 1. The sick bed 1 has a frame 2, attached to the underside of which are rollers 3 for ensuring the mobility of the sick bed 1.

In addition, the sick bed 1 comprises a bed 4 with a support surface 4′ and a foot area 4″ for a patient, with the bed 4 connected to the frame 2 via a connecting element 5.

The inventive sick bed 1 comprises a mobilisation unit that is schematically indicated by hydraulic cylinders 6. The hydraulic cylinders 6 are driven so as to place the foot area 4″ of the bed 4 in motion, e.g., to alternately raise and lower it. In this way, the legs of a patient lying on the bed 4 can be continuously moved and mobilised.

The connecting element 5 has connecting joints which can assist with changing the orientation and/or inclination of the connecting element 5 relative to the frame 2, and the orientation and/or inclination of the support surface 4′ of bed 4 relative to the frame 2. The orientation and/or the inclination are effected by a verticalisation device that is indicated by a drive 7 (such as a hydraulic cylinder) in the embodiment shown in FIG. 1. The drive 7 can change the inclination of the connecting element 5 relative to the carrier 2. It is obvious that an increase in the inclination, possibly to nearly the vertical, also brings the bed 4 into a more greatly inclined position. A patient lying on and fixed on the bed 4 is thus also placed in an upright position of up to 90° relative to the horizontal lying position. In addition, an inclination in the negative direction is possible by inclining the support surface such that the head area 4′ of the support surface 4 is lower than the foot area 4″. The patient's circulation can thus be influenced in different ways. It is also obvious that, in the event of verticalisation, the whole bed must be lowerable in order that therapeutic or nursing care may be performed on the patient.

According to the invention, the sick bed 1 has a controller or regulator for the drives 6, 7 which is indicated schematically by the box 8. The controller/regulator 8 can be attached anywhere on sick bed 1. It is also possible to arrange the controller/regulator 8 for the drives 6,7 at a remove from the sick bed, with the signals being transmitted to the actuators 6,7 via cable or wirelessly.

The input signals received by the controller/regulator 8 are physiological parameters for the patient lying on the bed 4, such as blood pressure, heart rate, device temperature, blood sugar, intracranial pressure, etc. These input parameters, such as pulse, blood pressure, etc., are measured by one or more measuring devices (not shown) and transmitted to the controller/regulator 8. Evaluation of the parameter indicates how the drives 6 and/or 7 need to be driven in order that the movement or orientation of the patient may influence specific physiological parameters (e.g. the input parameters) with a view to getting closer to a desired value. This control process is repeated at certain intervals in order that a value/curve of the patient's physiological parameters toward the setpoints may be achieved.

The measuring device and/or the controller/regulator 8 can also play a monitoring function. Monitoring of the physiological target parameters can also be carried out separately from the controller/regulator 8, however.

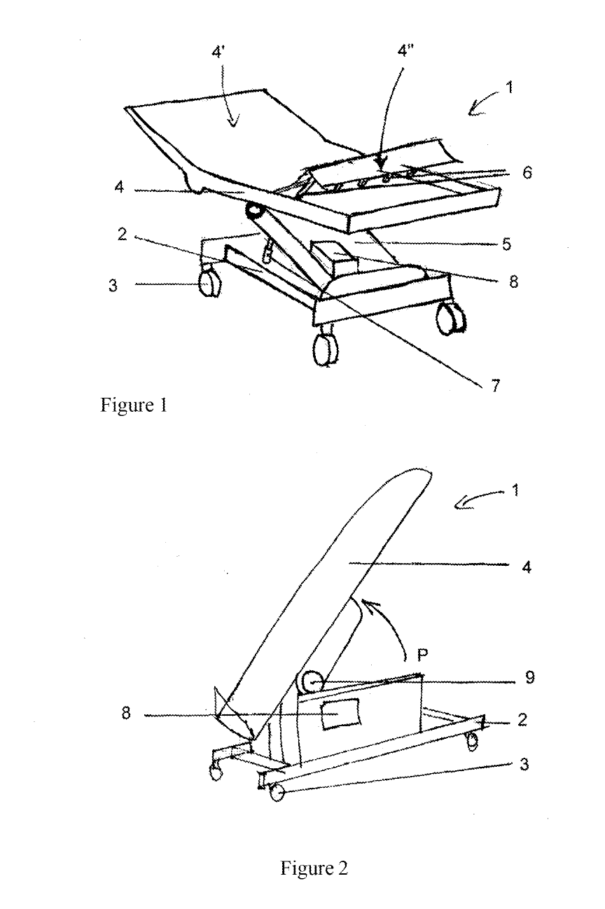

FIG. 2 shows a further embodiment of an inventive sick bed 1, with a frame 2 provided with rollers 3 and a bed 4. In the illustration of FIG. 2, the bed 4 is aligned at a relatively steep angle to the horizontal. The bed 4 is rotated about an axis 9 in a direction indicated by an arrow P, and consequently propped up. A patient strapped to the bed 4 will likewise be propped up. The verticalisation has effect on the physiological parameters of the same.

Also in this embodiment, the angle of inclination of the bed 4 relative to the horizontal is set by measured physiological parameters of a patient fixed on the bed 4. The angle to be set is determined at specific time intervals or continuously in a control circuit from the measured values with the help of the controller/regulator 8. In this way, the measured or other physiological parameters of a patient can be influenced in a targeted manner and changed to a specified target value and/or target curve.

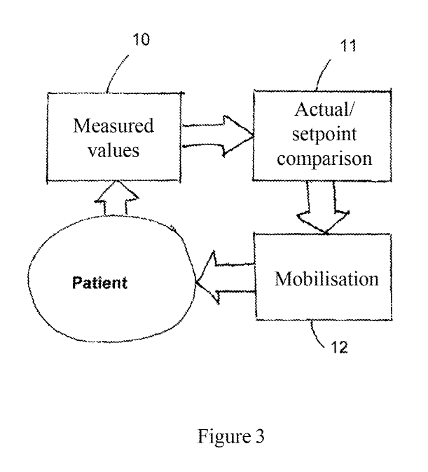

FIG. 3 schematically shows a control circuit, as used in the present invention. In the control system, measured values 10 are measured with the aid of a measuring device; the physiological parameters of the patient arranged on the support surface of the inventive sick bed are determined.

These measured values are made available for an actual/setpoint comparison 11. If the actual values differ from the setpoint values, the mobilisation 12 (or verticalisation) is changed so as to reduce the deviation of the actual values from the setpoint values.

In particular, the orientation of the bed and thus of the patient, the foot mobilisation of the patient, etc. can be changed accordingly.

With the described beds 1, a method or therapy concept with the goal of automated, continuous, patient-specific control/regulation of physiological patient parameters can be realised.

1. A patient bed comprising a frame, a bed connected to the frame and having a support surface for a patient, with at least one subsection of the bed arranged such that it can be moved relative to the frame, and at least one drive for setting the relative orientation of the at least one subsection of the bed relative to the frame, wherein
the patient bed has at least one of a controller and a regulator for activating the at least one drive as a function of at least one measured physiological parameter of the patient.

2. The patient bed in accordance with claim 1, wherein
the at least one drive is configured for setting the inclination of the support surface of the bed or a subsection of the support surface of the bed relative to the frame.

3. The patient bed in accordance with claim 2, wherein
the inclination can be adjusted to an angle approx. −30° and +90° with respect to the horizontal position of the support surface.

4. The patient bed in accordance with claim 3,
the inclination of the support surface can be adjusted such that the head area of the bed is inclined downwards.

5. The patient bed in accordance with claim 1, wherein
the at least one drive is configured for changing the orientation of a part of the support surface of the bed for the purpose of mobilising an area of the patient's body lying on the support surface.

6. The patient bed in accordance with claim 1, wherein
the patient bed comprises a measuring device for measuring the at least one physiological parameter of the patient, particularly a circulation parameter, such as heart rate, blood pressure, pulse, ECG, blood sugar, body temperature, intracranial pressure, etc.

7. The patient bed in accordance with claim 1, wherein
the controller and regulator, respectively, has at least one of a control unit, a simple regulator, and an automatic regulator.

8. The patient bed in accordance with claim 1, wherein
the controller/regulator is arranged at the patient bed.

9. The patient bed in accordance with claim 1, wherein
the patient bed is be equipped such that the support surface can be cooled, warmed, put into a harder or softer setting, and/or can convey sensory stimuli.

10. A monitoring and therapy device, comprising:
at least a patient bed comprising a frame, a bed connected to the frame and having a support surface for a patient, with at least one subsection of the bed arranged such that it can be moved relative to the frame, and at least one drive for setting the relative orientation of the at least one subsection of the bed relative to the frame; and
at least one of a controller and a regulator for activating the at least one drive as a function of at least one measured physiological parameter of the patient.

11. The monitoring and therapy device in accordance with claim 10, wherein
the bed is preferably connected to the frame such that the support surface can be inclined relative to the frame up to a predetermined angle.

12. The monitoring and therapy device in accordance with claim 10, wherein
the at least one drive can be configured for changing the orientation of a part of the support surface of the bed for the purpose of mobilising an area of the patient's body lying on the support surface.

13. The monitoring and therapy device in accordance with claim 10, wherein
the monitoring and therapy device includes a measuring device for measuring the at least one physiological parameter of the patient, particularly a circulation parameter, such as heart rate, blood pressure, pulse, as well as body temperature, intracranial pressure, etc.

14. A therapy concept for the human body, comprising the following steps:
a) measuring at least one physiological parameter of a patient, in particular by a measuring device, and
b) mobilising a body part of a patient and/or changing in the orientation of the patient's body as a function of the measured physiological parameter(s).

15. The therapy concept according to claim 14, wherein
step b) includes verticalising a body part of a patient in the range −90° and +90°.

16. The therapy concept according to claim 14, wherein
step a) includes measuring the at least one physiological parameter continuously or at discrete time intervals.

17. The therapy concept according to claim 14, wherein
step a) includes comparing the at least one physiological parameter with at least one predetermined setpoint value and determining a deviation between the physiological parameter and the setpoint value.

18. The therapy concept according to claim 17, wherein
the concept includes determining the intensity of the mobilisation and the angle of inclination of the body, respectively, such the deviation is counteracted.

19. The therapy concept according to claim 14, wherein
the concept includes an actual/setpoint comparison of the measured physiological parameter with a predetermined value.
